(19) United States
(12) Patent Application Publication
(10) Pub. No.: US 2024/0058008 A1
(43) Pub. Date: Feb. 22, 2024

(54) Anastomosis Seal Stent Assembly

(71) Applicant: Intraluminal Industries, LLC, Laredo, TX (US)

(72) Inventors: Michael Christopher Morris, Laredo, TX (US); Kenneth W. Horton, JR., South Glastonbury, CT (US); John Clark, Craughwell Co. (IE); Petrina Verdon, Ballinasloe Co. (IE); Sebastian Pintal, Galway (IE); Tom Feeney, Galway (IE)

(73) Assignee: Intraluminal Industries, LLc, Laredo, TX (US)

(21) Appl. No.: 17/892,193

(22) Filed: Aug. 22, 2022

(51) Int. Cl. A61B 17/12; A61B 17/11

(52) U.S. Cl. CPC A61B 17/12045; A61B 17/1114; A61B 17/12136; A61B 2017/1132

(57) ABSTRACT

A seal assembly for insertion into a hollow body organ is provided. The assembly includes a tubular hollow extrusion having a proximal end, a distal end and an interior surface between the proximal end and the distal end. A proximal balloon is located at the proximal end of the extrusion and a distal balloon is located at the distal end of the extrusion. An expandable scaffold engages the interior surface of the extrusion.

### BACKGROUND OF THE INVENTION

### Field of the Invention

The invention relates to a seal assembly to manage leaks and minimize the significance of a leak leaks at an anastomosis site.

### Description of the Related Art

A gastrointestinal resection is a surgical procedure to remove a portion of GI tract damaged by disease or trauma. During surgery, the diseased section is removed, and the two healthy ends are reattached (anastomosis).

These surgeries can result in leaks. While anastomotic leaks have been extensively studied, a successful anastomosis is dependent on multiple factors, including patient health, surgical technique, and the healing process (physiological, biochemical, and morphological factors).

Among postoperative complications, anastomotic leaks inflict more pain and suffering than any other surgical complication. Despite continual improvement in surgical techniques, anastomotic leaks associated with gastrointestinal surgical procedures remain a major cause of postoperative mortality and morbidity.

Colorectal resections for the treatment of cancer, diverticulitis, inflammatory bowel disease (IBD), or polyps are of particular interest because of the unacceptably high leak rates. When a leak is diagnosed, there are four main treatment options: antibiotics, drainage, fecal diversion, or takedown of the anastomosis (which results in a permanent colostomy).

Early diagnosis of an anastomotic leaks is crucial for the prevention of mortality. However anastomotic leaks are often insidious. Patients can experience a post-operative anastomotic leak but remain asymptomatic for days, resulting in a delayed diagnosis. A delayed diagnosis can lead to prolonged contamination of the abdominal, thoracic, or pelvic cavity, leading to severe sepsis and progression to multi organ failure and death.

Due to the relatively high propensity for colorectal leaks and the challenges of managing these leaks, colorectal surgeons often opt to proactively circumvent lower part of the colon with a diversion. This process entails looping a piece of the intestine through the abdominal wall and creating an artificial opening (diverting ostomy). The ostomy diverts fecal matter away from the lower GI tract into an external bag.

Diverting ostomies for low colorectal anastomosis has long been used as a method of “protecting” the anastomosis. Although several studies have demonstrated that there is no difference in the leak rate between patients who are diverted and those who are not, the consequences related to pelvic sepsis are certainly reduced with diversion.

Diverting ostomies and stoma-related complications remain common and are associated with significant morbidity as well as cost. Some of the most prevalent complications of stoma formation include peristomal skin complications, retraction, stomal necrosis, stomal stenosis, prolapse, bleeding, dehydration from high ostomy output, and parastomal hernia.

In summary, a diverting ostomy (and its subsequent reversal) is a major surgical procedure that carries a significant risk of complications. The occurrence of complications following surgery has significant health and financial consequences.

There is an unmet clinical need to:
Protect and isolate the anastomosis from luminal contents;
Prevent leakage into adjacent cavities;
Proactively monitor the anastomosis in real time;
Promote and accelerate the healing process; and
Reduce or eliminate the need of colostomies or ileostomies (for colorectal surgeries).

Until now, no device has satisfactorily addressed the needs of the surgeon, patient, and payors.

It would be beneficial to provide a device to significantly reduce the clinical and economic burdens of anastomotic leaks.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, a seal assembly for insertion into a hollow body organ is provided. The assembly includes a tubular hollow extrusion having a proximal end, a distal end and an interior surface between the proximal end and the distal end. A proximal balloon is located at the proximal end of the extrusion and a distal balloon is located at the distal end of the extrusion. An expandable scaffold engages the interior surface of the extrusion.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of an anastomosis seal assembly according to an exemplary embodiment of the present invention;

FIG. 2 is an end elevational view of the seal assembly of FIG. 1 showing the outer diameter of the body of the seal assembly;

FIG. 3 is an enlarged perspective view of the seal assembly of FIG. 1;

FIG. 3A is a sectional view of the seal assembly of FIG. 3, taken along lines 3A-3A;

FIG. 4 is an end elevational view of the seal assembly of FIG. 1, with a balloon inflated to show an exemplary outer diameter of the inflated balloon;

FIG. 5 is a perspective view of the seal assembly of FIG. 1 loaded onto a commercially available scaffold delivery catheter;

FIG. 6 is a side elevational view of the seal assembly of FIG. 1 in a simulated lumen of a body organ;

FIG. 7 is a sectional view of an alternative embodiment of a seal assembly according to the present invention;

FIG. 8 is a perspective view of the seal assembly of FIG. 1 attached to a distal end of a colonoscope for delivery to an anastomosis site;

FIG. 9 is a perspective view of the biomarkers placed on the seal assembly of FIG. 1;

FIG. 10 is a perspective view of the seal assembly of FIG. 1 delivering a drug; and

FIG. 11 is a perspective view of the seal assembly of FIG. 1 providing a tissue sealant.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import.

As used herein, the term “proximal” means a location closer to an inserting clinician and the term “distal” means a location farther from the inserting clinician. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Anastomotic leaks are inevitable, and early management is critical for the patient's welfare. The inventive dual balloon seal assembly is provided to protect and preserve the anastomosis; isolate the wound from luminal contents while preventing leakage into adjacent cavities; and utilize a controlled negative pressure to actively suction and drain the wound, which can accelerate the wound healing process by improving tissue perfusion, evacuating exudate, and facilitating the removal of bacteria from the wound.

Aspirated exudate is an indication of complications such as leaks, excess bleeding, and infection, which can lead a clinician to determine that post-operative treatment may be necessary. The inventive seal assembly can reduce the clinical and financial burdens of anastomotic leaks by protecting the anastomosis and promoting healing. The seal assembly combines endoscopic vacuum therapy (EVT) with self-expanding metal scaffold (SEMS) technology, while eliminating the shortcomings inherent in each. Because the inventive seal assembly is an intraluminal diversion device, the inventive seal assembly can be an alternative to temporary diverting ostomies.

Broadly, the inventive seal assembly that fuses multiple technologies of endoscopic vacuum therapy (EVT) with self-expanding metal scaffold (SEMS) technology into one easy-to-use, economically efficient device. The inventive seal assembly can address may of the clinical challenges of managing anastomotic leaks and promoting wound healing.

The inventive seal assembly is a flexible endoluminal dual balloon system that protects the anastomosis while actively promoting localized healing. The seal assembly has two balloons that are inflated by means of a syringe. Insufflation of the balloons anchor the assembly in place and establishes a seal about the anastomosis. Perforations between the two balloons enable negative pressure to be established between the balloons. This helps not only to anchor the system, but also to remove exudate from around the anastomotic site, all while administering negative wound pressure therapy to the wound.

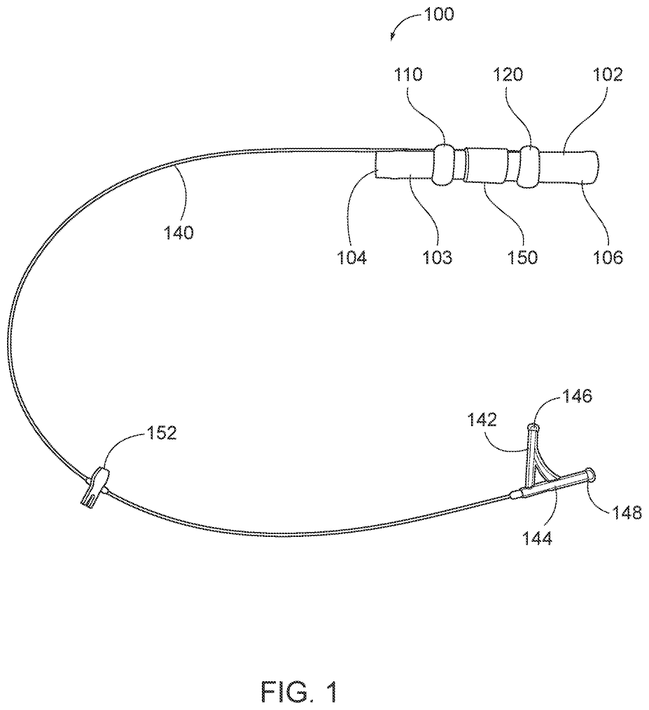

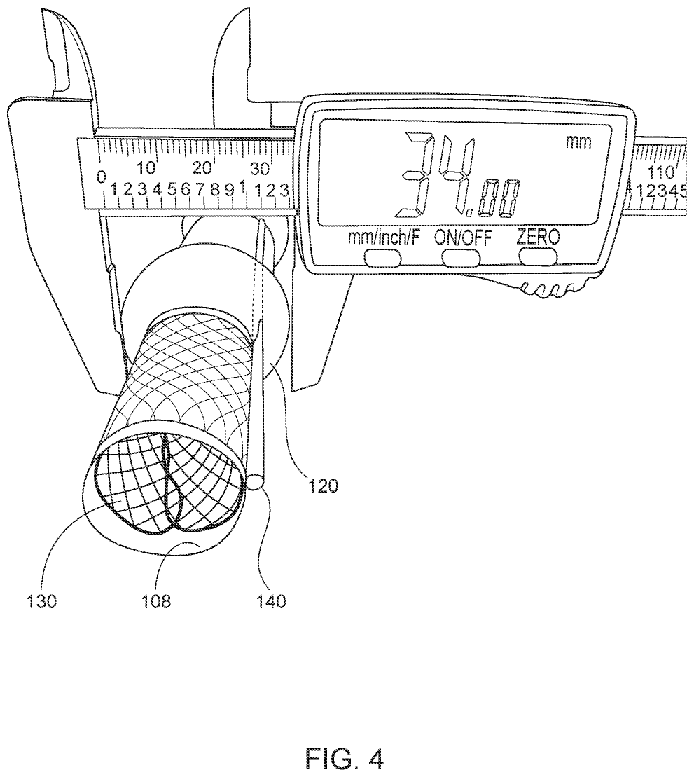

Referring to FIGS. 1-6, a seal assembly 100 for insertion into a hollow body organ 50 at an anastomotic site 52 is shown. Body organ 50 can be an intestinal tract or other body part that may involve an anastomosis procedure.

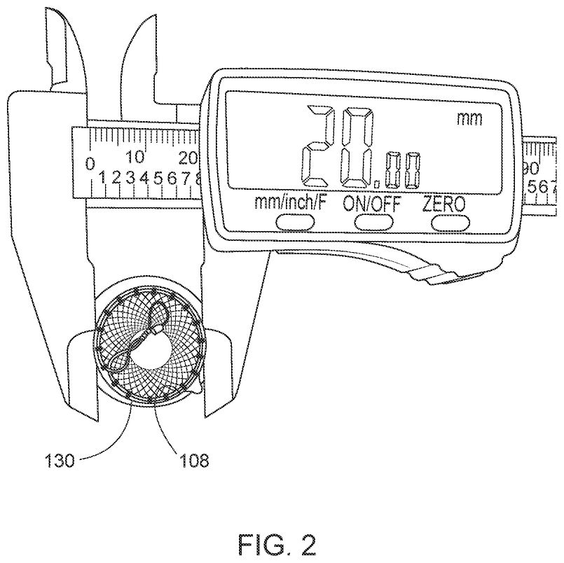

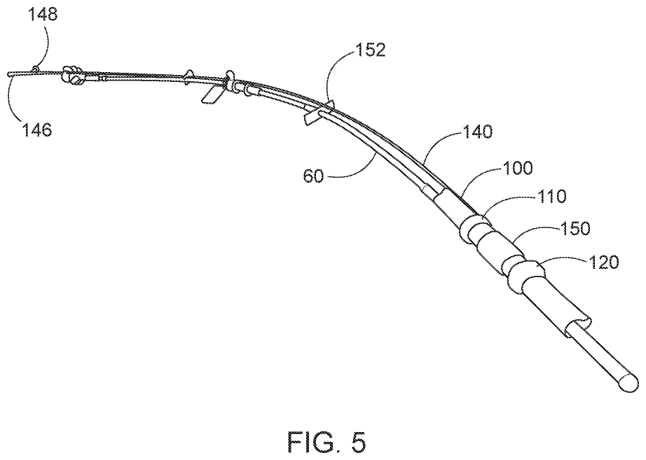

Seal assembly 100 includes a tubular hollow extrusion 102 having a proximal end 104, a distal end 106 and an interior surface 108 extending between the proximal end 104 and the distal end 106. A proximal balloon 110 is located at the proximal end 104 of the extrusion 102 and a distal balloon 120 is located at the distal end 106 of the extrusion 102. An expandable scaffold 130, shown in FIG. 2, is separate from extrusion 102 and is initially housed in a scaffold delivery catheter 60, shown in FIG. 5. In an exemplary embodiment, scaffold delivery catheter 60 can be an SX-Ella delivery system, manufactured by ELLA-CS of the Czech Republic. When assembly 100 is deployed, the scaffold delivery catheter 60 is withdrawn proximally from assembly 100, allowing scaffold 130 to expand, wherein scaffold 130 engages the interior surface 108 of the extrusion 102 and expands extrusion 102. The outer diameter of the scaffold 130 is sized to be just slightly smaller in diameter of the intestinal lumen in which assembly 100 is deployed.

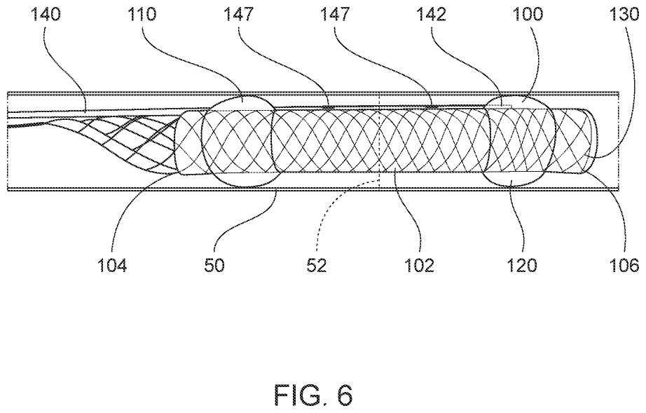

Each of the proximal balloon 110 and the distal balloon 120 is constructed from a compliant material and configured to conform and seal to an irregular lumen in body organ 50 and anchor the assembly 100 inside the lumen. See FIG. 6.

As shown in FIGS. 2-4, proximal balloon 110 and distal balloon 120 each have an expanded diameter of about 34 mm, Extrusion 102 has an exemplary expanded external diameter of about 20 mm and an exemplary expanded internal diameter of about 19 mm, although those skill in the art will recognize that the diameters can be other sizes as well. A distance between balloons 110, 120 can be about 65 mm, although those skilled in the art will recognize that the distance can be other than about 65 mm.

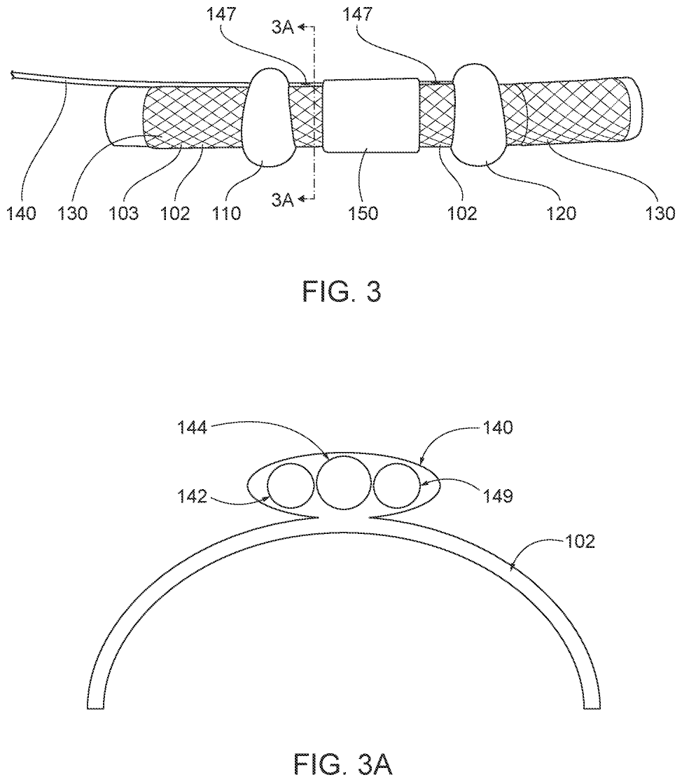

As shown in FIG. 3, scaffold 130 extends proximally of the proximal balloon 110 and also extends distally of the distal balloon 120. Scaffold 130 is a self-expanding scaffold and can be constructed of a known self-expanding material, such as Nitinol. Scaffold 130 is configured to prevent the body 102 from collapsing after inflation.

Referring back to FIG. 1, assembly 100 further includes a multi-lumen extrusion 140 extending along an exterior surface 103 of tubular hollow extrusion 102. Multi lumen extrusion 140 includes a balloon inflation lumen 142 and a vacuum lumen 144. A stiffening rod (not shown) can be inserted into vacuum lumen 144 to provide rigidity to assembly 100 to assist in inserting assembly 100.

Balloon inflation lumen 142 is in fluid communication with the proximal balloon 110 and also with the distal balloon 120. Balloon inflation lumen 142 transmits an inflation fluid, such as saline, from a proximal balloon inflation luer 146, to balloons 110, 120 to inflate balloons 110, 120 when assembly 100 is placed at anastomotic site 52.

Vacuum lumen 144 is in fluid communication with the exterior surface 103 of the extrusion 102 through a perforations 147 in vacuum lumen 144 between the proximal balloon 110 and the distal balloon 120. Perforations 147 provide for fluid communication between the external of extrusion 102 and between balloons 110, 120 and the body lumen 50 in which assembly 100 is inserted. Perforations 147 allow for a vacuum to be pulled through vacuum lumen 144 to enable negative pressure to be established exterior of extrusion 102 between the balloons 110, 120. This negative pressure helps not only to anchor the assembly 100, but also to remove exudate from around the anastomotic site 52, all while administering negative wound pressure therapy to the wound

Vacuum lumen 144 is in fluid communication with a vacuum luer 148 and is configured to pull a vacuum of about 20 mm Hg. When vacuum lumen 144 is drawing a vacuum, any exudate that may leak through anastomotic site 52 can be sucked into vacuum lumen 144 and visualized by a clinician, providing the clinician with an indication that a leak my be present ad the anastomotic site 52.

Assembly 100 further includes an open cell foam material 150 located on the exterior surface 103 of the extrusion 102 between the proximal balloon 110 and the distal balloon 120. Open cell foam material 150 can assist with wound evacuation. In simple terms, negative pressure wound therapy (NPWT) refers to any device that tightly seals the wound creating a near airtight environment to which a vacuum can be applied resulting in a series of biological reactions that enhance wound healing.

Three foam types are used in the VAC systems. Black polyurethane ether (VAC GranuFoam, KCI), a black polyurethane ester (VAC VeraFlow, KCI) and a white polyvinyl alcohol (VAC Vers-foam, KCI) foam, which has very small pore sizes and is used to protect critical structures without inducing microdeformations.

The black foam material is a hydrophobic, open-cell reticulated polyurethane (PU) foam that forms a structure resembling a three-dimensional net. This lattice formation deforms the wound surface and allows the vacuum to be evenly distributed throughout the foam and improves fluid drainage.

Microdeformation describes the mechanical changes that occur on the microscopic scale when suction is applied to the porous material resulting in an undulated wound surface. For PU foam interfaces, treating wounds for several days results in a cobble-stone appearance of the wound surface.

Microdeformation is believed to stimulate vessel sprouting towards the wound. This is described in further detail in the secondary effects of NPWT. Microdeformation causes localized hypoxia that causes an increase in local vascularity. Factors known to affect the efficiency of microdeformation include the level of suction, the pore size and the consistency of the foam, the tissue being treated and the deformability of the surrounding tissues.

As shown in FIG. 5, assembly 100 is configured for removable attachment to scaffold delivery catheter 60. A clip 152 is provided along multi lumen extrusion 140 between assembly 100 and luers 146, 148. Clip 152 is used to releasably secure and register assembly 100 to scaffold delivery catheter 60.

To use assembly 100, assembly 100 is slid over a distal end of scaffold delivery catheter 60, as shown in FIG. 5, with clip 152 being clipped to scaffold delivery catheter 60. Scaffold delivery catheter 60 with assembly 100 is inserted into the patient until distal balloon 130 is on one side of an anastomotic site 52 and proximal balloon 110 is on another side of anastomotic site 52. FIG. 6 shows an end-to-end anastomosis, although those skilled in the art will recognize that assembly 100 can be used with other types of anastomoses as well.

After assembly 100 is in place, clip 152 is released from scaffold delivery catheter 60 and scaffold delivery catheter 60 can be withdrawn. The shape memory material that comprises scaffold 130 expands to a natural condition, expanding scaffold 130 toward the inner wall of body organ 50 to expand extrusion 102 and allow body fluids to pass through extrusion 102 after assembly 100 is fully inserted into body organ 50.

Sealing fluid, such as saline, can be connected to balloon inflation luer 146 and pumped into balloon inflation luer 146, through balloon inflation lumen 142, and to balloons 110, 120 to inflate balloons 110, 120 and seal balloons 110, 120 against the inner wall of body organ 50.

The stiffening rod/guidewire is removed and a vacuum device (not shown) can be attached to vacuum luer 148 and a vacuum drawn through vacuum lumen 144. Any exudate or leakage from anastomotic site 52 can be sucked into vacuum lumen 144 though perforations 147 in extrusion 102, which can be visualized by a clinician to determine whether the anastomotic site 52 has a leak. In a first embodiment, the vacuum can be constant through vacuum lumen 144. In an alternative embodiment, however, the vacuum can be intermittent, as determined by the clinician.

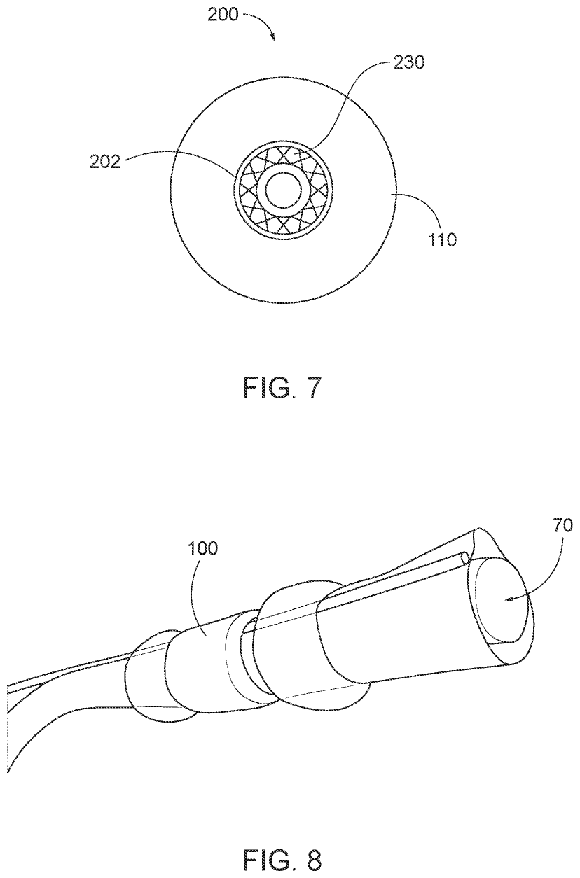

In an alternative embodiment of a seal assembly 200 according to the present invention, shown in FIG. 7, a scaffold 230 is integrally formed within extrusion 202.

Alternatively, instead of delivering seal assembly 100, 200 on scaffold delivery catheter 60, seal assembly 100, 200 can be delivered using a colonoscope 70, as shown in FIG. 8. With the integral scaffold 230 delivery option, the seal assembly 100, 200 can be delivered over a colonoscope, as shown in FIG. 8. With the independent scaffold 130, the seal assembly 100, 200 can either be delivered under fluoroscopy over the stent delivery system 60 or over the colonoscope 70.

Currently, early detection of leaks within the first postoperative days by clinical examination is difficult. Clinical indicators are neither sensitive nor specific. Postoperative clinical presentation (such as pain, fever, tachycardia, peritonitis) can be vague and often leads to delayed diagnosis and intervention.

Seal assemblies 100, 200 greatly reduce the clinical significance of leaks by actively protecting the anastomosis from luminal contents, drawing exudate from the localized site via vacuum, and actively promoting healing.

In recent years there have been significant advancements in the identification of diagnostic and predictive biomarkers of anastomotic leaks. This research is driven by the clinical need to identify patients at high risk of developing an anastomosis leak (“AL”) and to diagnose an AL earlier than current protocols allow. The ideal biomarker would allow for rapid, cost-effective and reliable prediction or detection of an AL in a time frame that allows clinicians to instigate interventions that minimize patient morbidity and mortality.

A clinically useful biomarker is one that is obtained non-invasively, is easily assayed and provides results that have high sensitivity and specificity. Broadly speaking, for anastomotic leaks, these can be biomarkers of ischemia, inflammation, tissue repair and the presence of bacterial contamination.

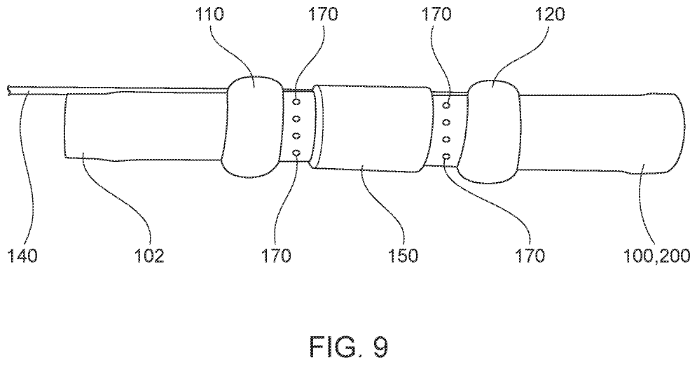

Referring to FIG. 9, implantable biomarkers 170 placed around the anastomotic site 52 could be incorporated into assemblies 100, 200 This concept would allow clinicians to continuously monitor peri-anastomotic biomarkers such as O₂, pH, C-reactive protein, lactate and pyruvate levels. This type of continuous monitoring system would help to identify at risk patients due to poor or deteriorating tissue oxygen tension (ptO₂). It would also allow clinicians the ability to assess the efficacy of interventions designed to improve intestinal ptO₂ and prevent a leak from occurring.

Biomarkers 170 can be wirelessly monitored via an RFID chip embedded in biomarker 170.

Gastrointestinal resection surgery is utilized for a number of diseases, but often is utilized to treat cancer. Depending on the stage of cancer development, combinations of two or more treatments are often recommended to achieve the best outcome. Surgery is the main treatment option wherein the tumor and adjacent lymph nodes are removed.

Secondary treatment options are used to eradicate any residual tumor at the surgical site, inhibiting the recurrence of the tumor and reducing the threat of distant metastasis. Pharmacotherapy, such as chemotherapy, is a typical secondary treatment option.

Localized drug delivery can lead to a decrease in drug-induced toxicity to healthy cells as a greater volume of the drug is localized in the colorectal cancer. This would benefit the patient because dose frequency and drug administration can be reduced.

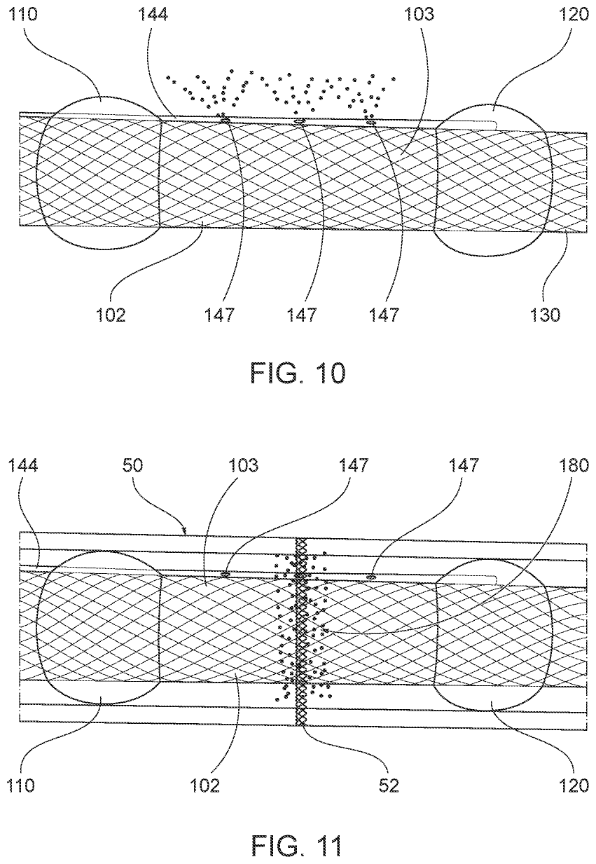

FIG. 10 shows how localized drug delivery can be effectuated using assemblies 100, 200. In lieu of drawing a vacuum through vacuum lumen 144, a drug can be administered through lumen 144 for delivery between balloons 110 120 through perforations 147 that are otherwise used to draw a vacuum from the anastomotic site. Alternatively, a third lumen 149, shown in FIG. 3A, can be provided for dedicated drug delivery.

Further, as with any surgical intervention, an anastomosis carries some risks, including inadequate seals to stop body fluid and air leakages. The use of adhesive biomaterials is an alternative for wound closure, due to the advantages that these adhesives offer over conventional approaches, including their simple and painless application and short implementation time.

Various types of surgical materials have been used for sealing and reconnecting tissues for numerous applications including anastomosis and sprayable adhesives for wound healing.

FIG. 11 shows an exemplary method of using assemblies 100, 200 to provide a tissue sealant 180. Sealant 180 can be applied to exterior surface 103 of extrusion 102 between balloons 110, 120. When scaffold 130 expands extrusion 102, prior to inflation of balloons 110, 120, sealant 180 engages the inner luminal wall 50 and is transferred to the inner luminal wall 50. When balloons 110, 120 are inflated, extrusion 102 is separated from luminal wall 50 and is more or less centered within luminal wall 50, but the sealant 180 remains on luminal wall 50 to seal luminal wall 50. Alternatively, the luminal wall area between balloons 110, 120 can be sprayed or flooded with sealant 180 via perforations 147 in vacuum lumen 144.

It will be further understood that various changes in the details, materials, and arrangements of the parts which have been described and illustrated in order to explain the nature of this invention may be made by those skilled in the art without departing from the scope of the invention as expressed in the following claims.

I claim:

1. A seal assembly for insertion into a hollow body organ, the assembly comprising:
(a) a tubular hollow extrusion having a proximal end, a distal end and an interior surface between the proximal end and the distal end;
(b) a proximal balloon at the proximal end of the extrusion;
(c) a distal balloon at the distal end of the extrusion; and

2. an expandable scaffold engaging the interior surface of the extrusion. The seal assembly according to claim 1, further comprising a supply lumen extending along the tubular hollow extrusion.

3. The seal assembly according to claim 2, wherein the supply lumen comprises a balloon inflation lumen and a vacuum lumen.

4. The seal assembly according to claim 3, wherein the balloon inflation lumen is in fluid communication with the proximal balloon and the distal balloon.

5. The seal assembly according to claim 3, wherein the vacuum lumen is in fluid communication with an exterior surface of the extrusion between the proximal balloon and the distal balloon.

6. The seal assembly according to claim 5, wherein the vacuum lumen is configured to pull a vacuum sufficient to provide a therapeutic effect.

7. The seal assembly according to claim 1, wherein the scaffold extends proximally of the proximal balloon.

8. The seal assembly according to claim 1, wherein the scaffold extends distally of the distal balloon.

9. The seal assembly according to claim 1, further comprising an open cell foam material on an exterior surface of the extrusion between the proximal balloon and the distal balloon.

10. The seal assembly according to claim 1, wherein the assembly is configured for removable attachment to a scaffold delivery catheter.

11. The seal assembly according to claim 1, wherein the scaffold comprises a self-expanding scaffold.

12. A seal assembly comprising:
(a) a tubular hollow extrusion having a proximal end, a distal end and an interior surface and an exterior surface between the proximal end and the distal end;
(b) a proximal balloon at the proximal end of the body;
(c) a distal balloon at the distal end of the body;
(d) an expandable scaffold engaging the interior surface of the extrusion;
(e) a balloon inflation lumen in fluid communication with the proximal balloon and with the distal balloon; and
(f) a vacuum lumen in fluid communication with the exterior surface.

13. The seal assembly according to claim 12, wherein the balloon inflation lumen and the vacuum lumen extend along a common supply lumen.

14. The seal assembly according to claim 12, wherein each of the proximal balloon and the distal balloon is constructed from a compliant material and configured to conform to an irregular lumen and anchor the assembly inside the lumen.

15. The seal assembly according to claim 12, wherein the scaffold is configured to prevent the extrusion from collapsing after balloon inflation.

16. The seal assembly according to claim 12, further comprising a biomarker attached to the exterior surface between the proximal balloon and the distal balloon.

17. The seal assembly according to claim 12, wherein the vacuum lumen is configured to deliver a drug.

18. The seal assembly according to claim 12, further comprising a drug delivery catheter configured to deliver a drug between the proximal balloon and the distal balloon.

19. The seal assembly according to claim 12, wherein the vacuum lumen is configured to deliver a sealant.

20. A seal assembly comprising:
(a) a tubular hollow extrusion having a proximal end and a distal end;
(b) a proximal balloon at the proximal end of the body;
(c) a distal balloon at the distal end of the body;
(d) a self-expanding scaffold engaging the extrusion;
(e) a balloon inflation lumen in fluid communication with the proximal balloon and with the distal balloon; and
(f) a vacuum lumen in fluid communication with the exterior surface and extending between the proximal balloon and the distal balloon, the vacuum lumen having at least one perforation formed therein.
